(19) United States
(12) Patent Application Publication
(10) Pub. No.: US 2020/0321103 A1
(43) Pub. Date: Oct. 8, 2020

(54) COMPUTER READABLE MEDIUM STORING INSTRUCTIONS FOR HEALTH MANAGEMENT SUPPORT, METHOD FOR HEALTH MANAGEMENT SUPPORT, AND SYSTEM FOR HEALTH MANAGEMENT SUPPORT

(71) Applicant: UNICHARM Corporation, Ehime (JP)

(72) Inventors: Yuya Suzuki, Kagawa (JP); Toru Sasaki, Kagawa (JP); Hiroki Ishikawa, Kagawa (JP); Akiko Tange, Kagawa (JP); Megumi Mimuro, Kagawa (JP); Takahiro Imagawa, Kagawa (JP); Eiji Shibaya, Kagawa (JP)

(73) Assignee: UNICHARM Corporation, Ehime (JP)

(21) Appl. No.: 16/908,004

(22) Filed: Jun. 22, 2020

Related U.S. Application Data
(63) Continuation of application No. PCT/JP2017/046123, filed on Dec. 22, 2017.

(51) Int. Cl. G16H 30/40; G16H 50/30; G16H 50/20; G06T 7/00; G06T 7/90

(52) U.S. Cl. CPC G16H 30/40; G16H 50/30; G06T 7/90; G06T 7/0014; G16H 50/20

(57) ABSTRACT

A non-transitory computer readable medium stores executable instructions that cause a computer to execute: transmitting, to a server, an image of an absorbent article containing vaginal discharge of a user; receiving, from the server, health information that is obtained from the image and indicates a health condition of the user; and displaying, on a display, a health information screen based on the health information. The health information screen displays a first message that indicates the health condition and a second message for improving the health condition.

### BACKGROUND

### Technical Field

The present invention relates to a computer readable medium storing instructions for health management support, a method for health management support, and a system for health management support.

### Description of the Related Art

Typically, vaginal discharge (vaginal secretion) such as menstrual blood discharged from the vagina of a woman changes depending on the health condition. Thus, as illustrated in Patent Document 1, menstrual blood on a sanitary product is extracted, and the health condition of the sanitary product user is analyzed.

### Patent Literature

[Patent Document 1] U.S. Patent Application Publication No. 2016/0324506

However, the technology disclosed in Patent Document 1 requires a dedicated system for extracting the menstrual blood on the sanitary product or the like. Thus, it has been difficult for the user to manage their health easily on the basis of information of the menstrual blood or the like.

### SUMMARY

One or more embodiments of the present invention provide a computer readable medium storing instructions for health management support, a method for health management support, and a system for health management support.

One or more embodiments of the present invention provide a computer readable medium storing instructions that cause a computer to execute:
a transmission process for transmitting, to a server, an image of an absorbent article to which vaginal discharge of a user is attached (i.e., absorbent article containing vaginal discharge);
a reception process for receiving health information from the server,
   the health information being information about a health condition of the user,
   the health information being obtained on the basis of the image; and
a health-information displaying process for displaying on a display a health information screen on the basis of the health information,
   the health information screen including a first message and a second message,
   the first message indicating the user's health condition, and
   the second message for improving the health condition.

According to one or more embodiments of the present invention, the user can manage their health easily on the basis of information of vaginal discharge.

### BRIEF DESCRIPTION OF THE DRAWINGS

FIG. 1 illustrates a terminal 10 and a server 11 according to one or more embodiments.

FIG. 2 illustrates a flow of a method for health management support according to one or more embodiments.

FIG. 3 illustrates details of a menses-information process according to one or more embodiments.

FIG. 4 illustrates a message image displayed on a display 23 when an image of a napkin is captured according to one or more embodiments.

FIG. 5 illustrates an example of a napkin image 210 according to one or more embodiments.

FIG. 6 illustrates a message image displayed on the display 23 when menses information is input according to one or more embodiments.

FIG. 7 illustrates an example of a determination result of the server 11 according to one or more embodiments.

FIG. 8 illustrates an example of a health information screen 27 according to one or more embodiments.

### DETAILED DESCRIPTION OF EMBODIMENTS

At least the following matters will become clear with the description of this specification and the attached drawings.

A computer readable medium according to one or more embodiments stores instructions that cause a computer to execute:
a transmission process for transmitting, to a server, an image of an absorbent article to which vaginal discharge of a user is attached;
a reception process for receiving health information from the server,
   the health information being information about a health condition of the user,
   the health information being obtained on the basis of the image; and
a health-information displaying process for displaying on a display a health information screen on the basis of the health information,
   the health information screen including a first message and a second message,
   the first message indicating the user's health condition, and
   the second message for improving the health condition.

According to one or more embodiments, the user is able to know about and improve their health condition on the basis of the information displayed on the health information screen, thereby managing their health easily.

In one or more embodiments,

the vaginal discharge includes menstrual blood, and

the instructions cause the computer to further execute:
an instruction displaying process for displaying a third message on the display,
the third message being for prompting to capture an image of the absorbent article when a predetermined number of days has passed since start of a menstruation of the user.

According to one or more embodiments, the user is able to capture an image of a napkin when the predetermined number of days has passed since start of a menstruation (e.g., a napkin on the third day since the start of the menstruation), that is a timing when typically the menstrual blood volume becomes stable. Thus, the user is provided with highly accurate information about their health condition.

In one or more embodiments,
the third message is displayed for prompting to capture an image of the absorbent article in a morning of a fourth day since the start of the menstruation, and
the absorbent article is one that has been used overnight on a third day since the start of the menstruation.

According to one or more embodiments, at the same timing in each menstruation, the user is able to capture an image of a napkin used. Thus, the user is able to easily know about a monthly change in their health condition.

In one or more embodiments,
in the transmission process, user information is further transmitted to the server,
the user information is information about the user, and
the health information is obtained on the basis of the image and the user information.

According to one or more embodiments, the user is provided with advice based on user's medical-interview information as well as based on the image of the absorbent article to which menstrual blood or the like is attached. This improves the accuracy of the advice for health management.

In one or more embodiments,
the vaginal discharge includes menstrual blood,
the instructions cause the computer to further execute:
   an input-screen displaying process for displaying an input screen on the display,
      the input screen being for inputting menstruation information,
      the menstruation information being information about menstruation when the image of the absorbent article is captured; and
   a menstruation-information reception process for receiving the menstruation information that is input,
in the transmission process, the menstruation information that has been received is further transmitted to the server, and
the health information is obtained on the basis of the image and the menstruation information.

According to one or more embodiments, the user is provided with advice based on the information of user's menstruation as well as based on the image of the absorbent article to which menstrual blood or the like is attached. This improves the accuracy of the advice for health management.

In one or more embodiments,
the vaginal discharge includes menstrual blood,
the instructions cause the computer to further execute:
   an input-screen displaying process for displaying an input screen on the display,
      the input screen being for inputting menstruation information,
      the menstruation information being information about menstruation when the image of the absorbent article is captured; and
   a menstruation-information reception process for receiving the menstruation information that is input,
in the transmission process, user information about the user and the menstruation information that has been received are further transmitted to the server, and
the health information is obtained on the basis of the image, the user information, and the menstruation information.

According to one or more embodiments, the user is provided with advice based on user's medical-interview information and user's menstruation information as well as based on the image of the absorbent article to which menstrual blood or the like is attached. This improves the accuracy of the advice for health management.

In one or more embodiments,
the menstruation information includes information indicating a number of absorbent articles that have been used by the user before capturing the image of the absorbent article, and
the input screen includes a fourth message that prompts the user to input the number of absorbent articles.

According to one or more embodiments, the number of used absorbent articles, which is in accordance with a menstrual blood volume of the user, is input to the computer as objective information about the menstruation.

In one or more embodiments,
the menstruation information includes information indicating color of the menstrual blood attached to the absorbent article, and
the input screen includes a plurality of colors and a fifth message,
   the fifth message being for making a user select a darkest color of the menstrual blood attached to the absorbent article from the plurality of colors.

According to one or more embodiments, the information about the color of the menstrual blood is input to the computer as well as the image of the absorbent article to which menstrual blood or the like is attached. This makes it possible for the user to be provided with high-quality advice even if there is an issue with the method for capturing the image of the absorbent article to which menstrual blood is attached, the image capturing environment, the quality of the image of the absorbent article, and the like.

In one or more embodiments,
the menstruation information includes information indicating whether there is any clotted matter in the vaginal discharge, and
the input screen includes a sixth message that prompts the user to input whether there is any clotted matter in the vaginal discharge.

According to one or more embodiments, the information indicating whether there is any clotted matter in the vaginal discharge is input to the computer as well as the image of the absorbent article to which menstrual blood or the like is attached. This makes it possible for the user to be provided with high-quality advice even if it is difficult to determine in the image of the absorbent article whether there is any clotted matter in the vaginal discharge.

In one or more embodiments,
the instructions cause the computer to further execute a field-instruction displaying process for displaying a field instruction image on the display,
   the field instruction image being an image for prompting to capture an image of a to-be-imaged absorbent article so that the image includes a portion of the to-be-imaged absorbent article where no menstrual blood is attached.

According to one or more embodiments, the user is able to capture the entire image of menstrual blood attached to the absorbent article.

This makes it possible to provide the user with higher-quality advice, compared with a case, for example, where an image of only a portion of the menstrual blood attached to the absorbent article is captured.

In one or more embodiments,
the first message is a message about stress or hormone balance.

According to one or more embodiments, the user is able to obtain knowledge about their stress level or hormone imbalance.

In one or more embodiments,
the second message is a message about a nutrient that the user should take.

According to one or more embodiments, the user is able to improve their health condition by having appropriate meals in accordance with the content of the message.

In one or more embodiments,
the health information is information about stress or hormone balance,
the instructions cause the computer to further execute
   a generating process for generating information indicating the first message and the second message on the basis of the health information.

According to one or more embodiments, the computer generates the messages, and thus, the server processes and burden can be reduced.

A method for health management support according to one or more embodiments, includes:
a transmission step in which a computer transmits to a server an image of an absorbent article to which vaginal discharge of a user is attached;
a reception step in which the computer receives health information from the server,
   the health information being information about a health condition of the user,
   the health information being obtained on the basis of information transmitted from the computer; and
a health-information displaying step in which the computer displays on a display a health information screen on the basis of the health information,
   the health information screen including a first message and a second message,
   the first message indicating the user's health condition,
   the second message for improving the health condition.

According to such a method, the user is able to know about and improve their health condition on the basis of the information displayed on the health information screen, thereby managing their health easily.

A computer system according to one or more embodiments includes:
a transmission module for transmitting, to a server, an image of an absorbent article to which vaginal discharge of a user is attached;
a reception module for receiving health information from the server,
   the health information being information about a health condition of the user,
   the health information being obtained on the basis of information transmitted from the computer; and
a health-information displaying module for displaying on a display a health information screen on the basis of the health information,
   the health information screen including a first message and a second message,
   the first message indicating the user's health condition,
   the second message for improving the health condition.

According to such a computer system, the user is able to know about and improve their health condition on the basis of the information displayed on the health information screen, thereby managing their health easily.

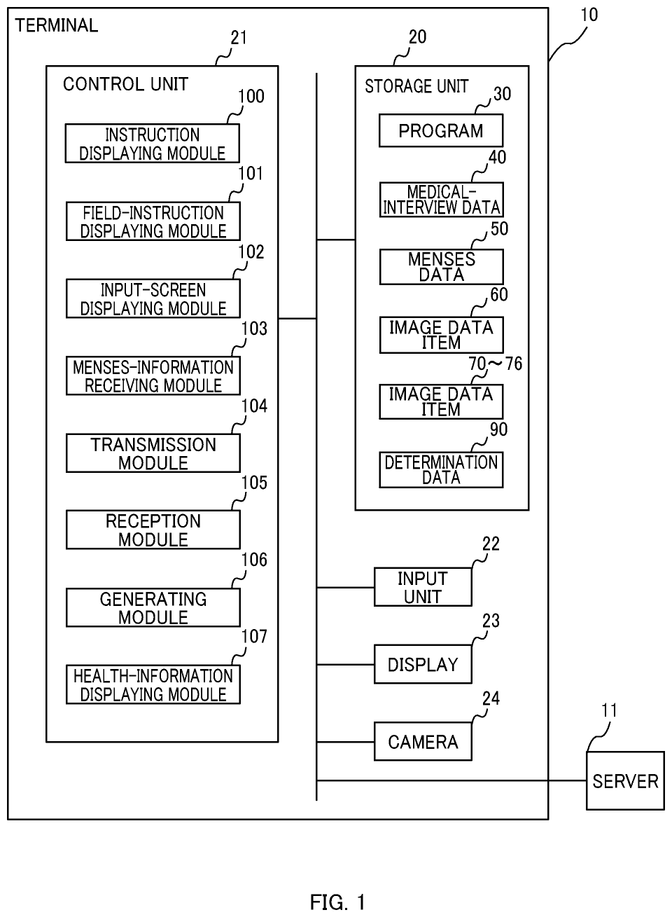

FIG. 1 illustrates a terminal 10 and a server 11. The terminal 10 is a smartphone or tablet, and implements various functions. In addition, by performing various processing, the terminal 10 displays, on a display 23 of the terminal 10, advice about the health condition of a user of the terminal 10 (hereinafter simply referred to as “user”) or the like.

The server 11 is an apparatus to which the terminal 10 is connected via a network and transmits or receives various types of information to or from the terminal 10.

The terminal 10 (computer) includes a storage unit 20, a control unit 21 (i.e., a controller), an input unit 22, the display 23, and a camera 24.

The storage unit 20 stores a program (i.e., instructions) 30 executed by a processor (not illustrated) of the terminal 10, medical-interview data 40, menses data 50, an image data item 60, image data items 70 to 76, and determination data 90. The program 30 is used when supporting the user's health management.

The medical-interview data 40 (user information) is data indicating “medical-interview information” of the user and includes information about a profile, lifestyle, and menses and condition. Herein, the information about a profile is information about, for example, the user's age, height, weight, and blood type, and whether the user has any children. The information about lifestyle is information about, for example, having balanced meals, good quality sleep, and a stress relief method. The information about menses and condition is information about, for example, a menstrual cycle, the number of menstrual days, menstrual blood volume, period pain, and premenstrual symptoms.

The menses data 50 (menstruation information) is data indicating “menses information” about the period (menstruation) of the user.

The image data item 60 is data indicating an image of a sanitary napkin (hereinafter simply referred to as “napkin (absorbent article)”) to which the menstrual blood (vaginal discharge) of the user is attached.

Each of the image data items 70 to 76 is data indicating an image (e.g., message image or illustration image) to be displayed as appropriate on the display 23 of the terminal 10 when the program 30 is executed.

The determination data 90 (health information) is data about the user's health condition obtained by processing of the server 11. Note that the server 11 executes instructions to determine the user's health condition on the basis of the medical-interview data 40, the menses data 50, and the image data item 60. Herein, a storage unit (not illustrated) of the server 11 stores a large number of information items indicating determination results of the user's health condition that a physician has determined on the basis of the “image data item 60”, the “menses information”, and the “medical-interview information” (that is, previously accumulated information items of the user). The server 11 performs pattern matching or the like by using the accumulated information items, generating the “determination data 90” indicating the user's health condition.

Although the determination data 90 herein is generated by the server 11 performing processing, the determination data 90 is not limited to this. For example, upon receiving the medical-interview data 40, the menses data 50, and the image data item 60, the server 11 displays, on a monitor (not illustrated), the user's “medical-interview information” and “menses information” and a napkin image 210 on the basis of the above data items. On the basis of these displayed information items, a physician may determine the user's health condition (stress or hormone balance) and input the determination data 90 to the server 11.

The control unit 21 is a functional block which is implemented in the terminal 10 by a processor (not illustrated) provided in the terminal 10 executing the program 30. The control unit 21 controls the entire terminal 10 and includes a plurality of blocks that execute various processing. Note that details of functional blocks implemented by the control unit 21 will be described later.

An operation result of the terminal 10 by the user is input to the input unit 22. The input unit 22 is implemented as a tap operation on the touch-panel display 23 of the terminal 10.

The display 23 displays various types of information on the basis of a command from the control unit 21 or an operation result by the user. The camera 24 obtains image data by capturing an image.

Functional Blocks Implemented in Control Unit 21

The control unit 21 includes an instruction displaying module 100, a field-instruction displaying module 101, an input-screen displaying module 102, a menses-information receiving module 103, a transmission module 104, a reception module 105, a generating module 106, and a health-information displaying module 107.

The instruction displaying module 100 performs a processing for displaying, on the display 23, a message image (described later) for prompting the user to capture an image of a napkin that is used overnight on the third day; the third day of a menstruation is a day when two days (predetermined number of days) have lapsed from the start of a period (start of menstruation) and typically the menstrual blood volume becomes stable.

The field-instruction displaying module 101 performs a processing for displaying, on the display 23, a field-instruction image (described later) for capturing an image of a napkin that is an image capturing target, so as to instruct for the user to include a portion of the napkin where no menstrual blood is attached (margin on the surface of the napkin).

The input-screen displaying module 102 performs a processing for displaying, on the display 23, an input screen (described later) for inputting the user's “menses information”. The menses-information receiving module 103 performs a processing for receiving the “menses information” that has been input.

The transmission module 104 transmits, to the server 11, the medical-interview data 40, the menses data 50, and the image data item 60, which are stored in the storage unit 20. The reception module 105 receives the determination data 90 from the server 11 and stores the determination data 90 in the storage unit 20.

The generating module 106 generates various message images (described later) about the user's health condition, on the basis of the determination data 90 about the user's health condition.

The health-information displaying module 107 performs a processing for displaying, on the display 23, a message image related to the health condition generated by the generating module 106.

Method for Health Management Support

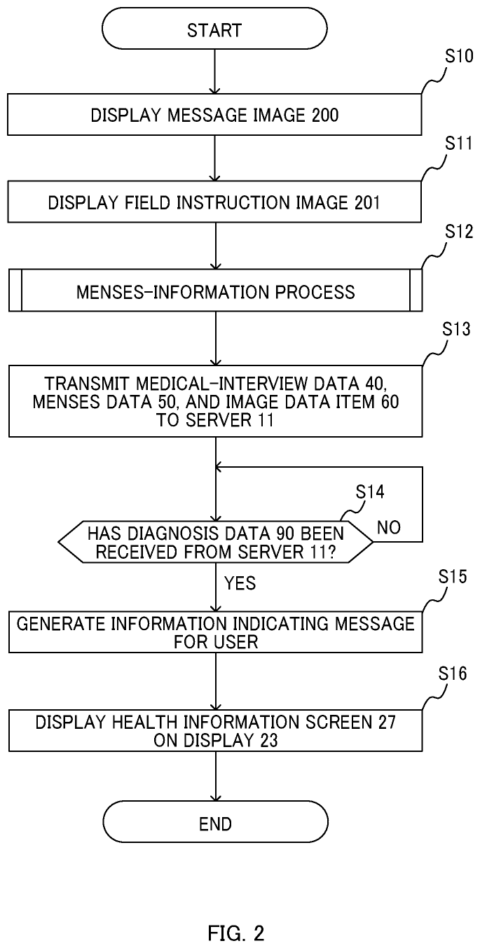

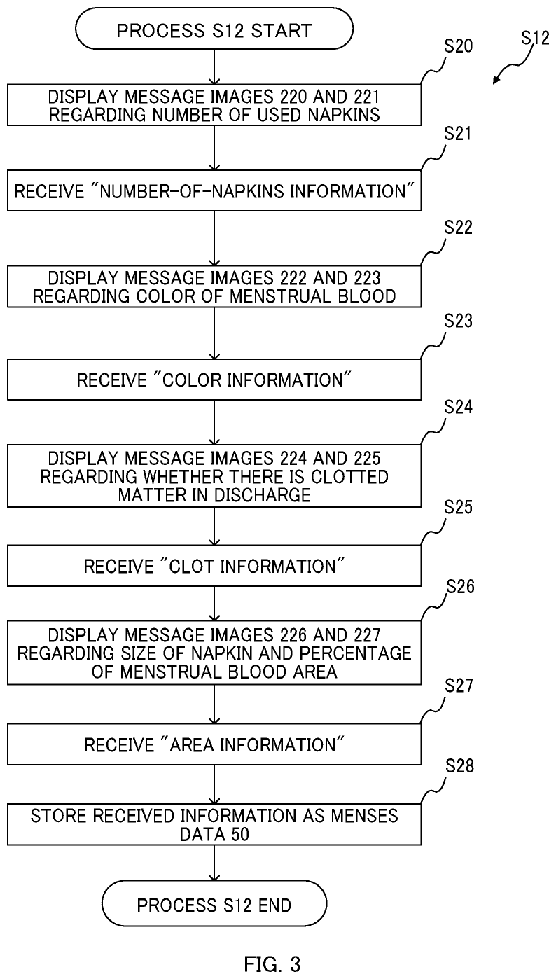

Now, a flow of a method for health management support will be described with reference to FIG. 2 and FIG. 3. Herein, the program 30 has been started as a result of an operation on the terminal 10 by the user.

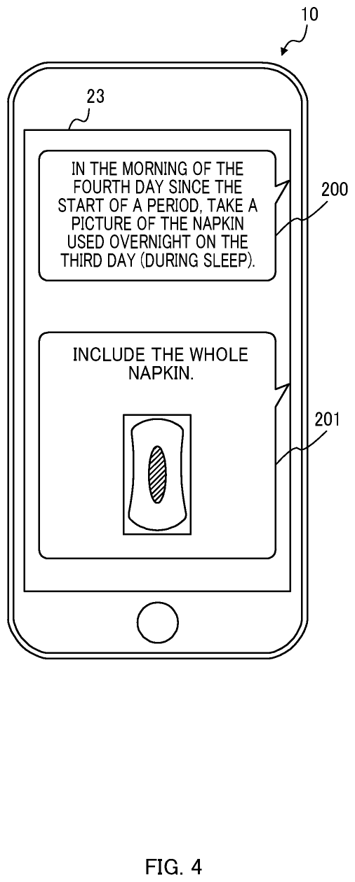

When the user operates a main screen (not illustrated) displayed on the display 23, the instruction displaying module 100 displays an image illustrated in FIG. 4 on the display 23.

Specifically, the instruction displaying module 100 displays, on the display 23 and on the basis of the image data item 70, a message image 200 (third message) for capturing an image of a napkin that is used overnight on the third day (S10: instruction displaying process). Note that the message image 200 indicates “In the morning of the fourth day since the start of a period, take a picture of the napkin used overnight on the third day (during sleep)”. Typically, the menstrual blood volume becomes stable at night on the third day since the start of a period. Thus, the user captures an image of the used napkin in the morning of the fourth day, thereby obtaining an image at a time suitable for deteunination. In addition, the user captures an image of a napkin at the same time (in the morning of the fourth day) in each month, and thus, the user can easily know a change in their health condition.

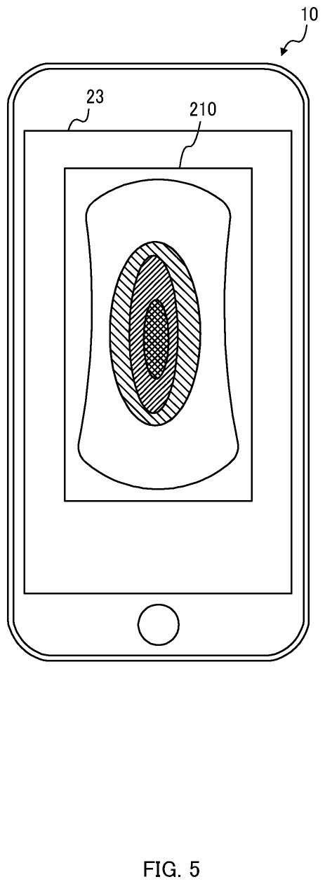

Subsequently, the field-instruction displaying module 101 displays, on the display 23 and on the basis of the image data item 71, a field instruction image 201 as an instruction of a field for capturing an image of a napkin (S11: field-instruction displaying process). Note that the field instruction image 201 includes the message “Include the whole napkin” and a reference image of the image capturing target. In the morning of the fourth day, when the user operates the terminal 10 to capture an image of a napkin used during sleep on the third day, an image of the napkin to which the menstrual blood is attached (hereinafter, the napkin image 210) illustrated in FIG. 5 is stored as the image data item 60 in the storage unit 20.

Note that, for example, if the user captures an image of only part of the menstrual blood attached to the napkin, the amount of information about the color and volume of the menstrual blood in the image, which is important for determination, is small. However, in one or more embodiments, since the field instruction image 201 is displayed, the user can capture an image suitable for determination.

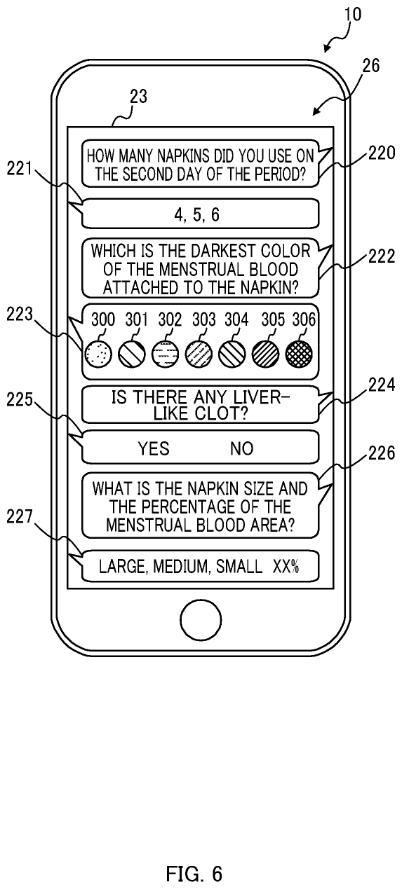

FIG. 3 is a flowchart illustrating an example of a process performed in a menses-information process (S12), and FIG. 6 illustrates an example of an input screen 26 displayed on the display 23.

First, as illustrated in FIG. 6, the input-screen displaying module 102 displays, on the display 23 and on the basis of the image data item 72, a message image 220 (fourth message) that prompts the user to input the number of napkins used since the start to the second day of a period and an image 221 of a plurality of choices corresponding to the number of used napkins (S20: input-screen displaying process). The message image 220 indicates “How many napkins did you use on the second day of the period?”, and the image 221 indicates “4, 5, 6”.

If the user selects “5” by tapping the display 23, for example, the menses-information receiving module 103 receives the selection result as “number-of-napkins information” (S21: menstruation-information reception process).

The input-screen displaying module 102 displays, on the display 23 and on the basis of the image data item 73, a message image 222 (fifth message) for making a user select the darkest color of the menstrual blood attached to the napkin and an image 223 indicating a plurality of colors (S22: input-screen displaying process). Note that the message image 222 indicates “Which is the darkest color of the menstrual blood attached to the napkin?”, and the image 223 indicates seven colors 300 to 306. In the image 223 herein, the color becomes darker toward the right side (the color 306 side). Thus, among the colors 300 to 306, the color 300 is the lightest, and the color 306 is the darkest.

When the user selects the color 306 by tapping the display 23, for example, the menses-information receiving module 103 receives the selection result as “color information” (S23: menstruation-information reception process).

The input-screen displaying module 102 displays, on the display 23 and on the basis of the image data item 74, a message image 224 (sixth message) that prompts the user to input whether there is any clotted matter in the vaginal discharge and a message image 225 indicating choices (S24: input-screen displaying process). Note that the message image 224 indicates “Is there any liver-like clot?”, and the message image 225 indicates “Yes, No”.

If the user selects “Yes” by tapping the display 23, for example, the menses-information receiving module 103 receives the selection result as “clot information” (S25: menstruation-information reception process).

The input-screen displaying module 102 displays, on the display 23 and on the basis of the image data item 75, a message image 226 that prompts the user to input the size of the napkin and a state of dispersion (area) of the menstrual blood. Also, the input-screen displaying module 102 displays a message image 227 that prompts the user to input (select) such information (S26). Note that the message image 226 indicates “What is the napkin size and the percentage of the menstrual blood area?”, and the message image 227 indicates “Large, Medium, Small, xx %”.

If the user selects “Medium” and inputs “50%” by tapping the display 23, for example, the menses-information receiving module 103 receives the input results as “area information” (S27). Note that the message image 226 may indicate choices “Large, Medium, Small, less than 30% of the whole napkin area, 30% to less than 50%, 50% to less than 80%, more than 80%”, for example. Alternatively, as the “area information”, for example, the menses-information receiving module 103 may receive the menstrual blood area traced in the napkin image 210 by the user with a finger. The napkin size is selected herein, but without limitation to this, for example, the usage or size (e.g., daytime xx cm or nighttime xx cm) of the napkin may be selected or input.

The menses-information receiving module 103 stores the received “number-of-napkins information”, “color information”, “clot information”, and “area information” as the menses data 50 in the storage unit 20.

When step S12 ends, the transmission module 104 transmits to the server 11 the medical-interview data 40, the menses data 50, and the image data item 60, which have been stored in the storage unit 20 (S13: transmission process). Upon receiving the medical-interview data 40, the menses data 50, and the image data item 60, the server 11 displays, on a monitor (not illustrated), the user's “medical-interview information” and “menses information” and the napkin image 210 on the basis of the above data items. On the basis of these displayed information items, the server 11 determines the user's health condition (stress or hormone balance).

Herein, the server 11 refers to the “number-of-napkins information”, the “color information”, the “clot information”, and the “area information” included in the “menses information” for determination. The “number-of-napkins information” is information indicating the menstrual blood volume from the start to the second day of a period of the user as a quantity of the “number of napkins”. And the “area information” is also information indicating the menstrual blood volume of the user as a quantity. Thus, if menorrhagia is determined on the basis of these information items, it is determined that the user may possibly have a symptom of anemia or the like. Note that the server 11 can determine the symptom with high accuracy because when running the predetermined instructions referred to are the “number of napkins” used and the actual area in which the menstrual blood is attached. In addition, when capturing the image of the napkin to which the menstrual blood is attached, the color in the image may change depending on uneven light on the napkin, reflection of light, characteristics of the camera 24, and the like. Thus, typically, it is difficult to accurately determine the color of the menstrual blood attached to the napkin. However, in one or more embodiments, the server 11 can refer to the “color information” about the color of the menstrual blood selected by the user by visual inspection. Thus, the amount of information that is input to the server 11 is increased, and the color of the menstrual blood can be determined more accurately. In addition, depending on the volume or color of the menstrual blood attached to the napkin, it is in some cases difficult to determine whether a liver-like clot is included in the image. In one or more embodiments, the server 11 can refer to the “clot information” indicating whether the liver-like clot is included, and thereby the determination accuracy is increased.

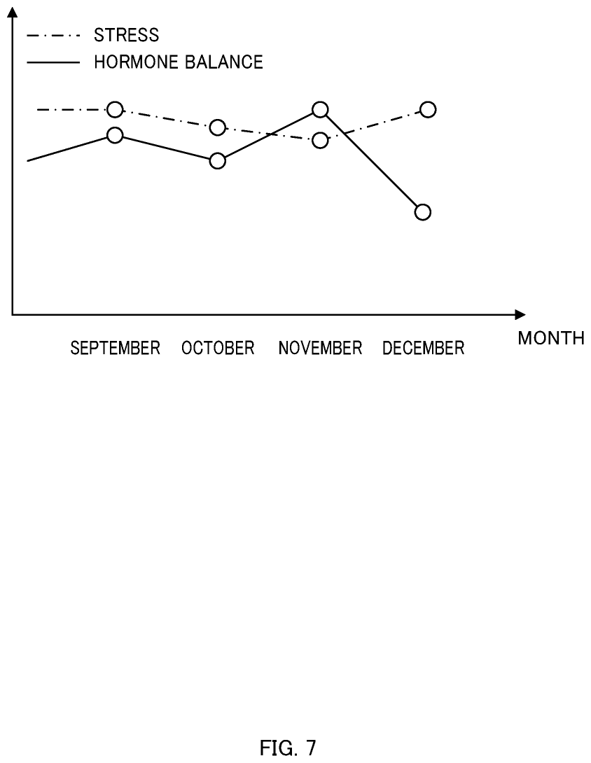

FIG. 7 illustrates an example of the “determination result” of the server 11. The server 11 quantifies “stress” and “ho lone balance” and records them as the “determination result” in a storage unit (not illustrated) of the server 11. Note that the chained line in FIG. 7 represents “stress” of the user, and the solid line represents “hormone balance” of the user. Herein, as the user's stress is smaller, the value of “stress” is increased, and as the user's hormone balance is better, the value of “hormone balance” is increased. The server 11 herein, for example, updates “stress” and “hormone balance” in December.

Subsequently, the server 11 transmits the “determination result” about the user's health condition as the determination data 90 to the terminal 10. The reception module 105 of the terminal 10 determines whether the determination data 90 has been received from the server 11 (S14). When the determination data 90 is received from the server 11 (S14: Yes, reception process), the generating module 106 generates information (the image data item 76) indicating a message for the user, on the basis of the determination data 90 (S15: generating process).

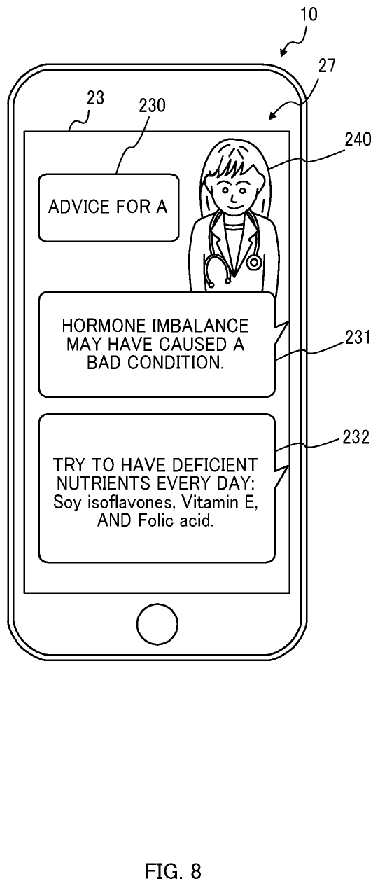

In addition, on the basis of the information generated in step S15, the health-information displaying module 107 displays, on the display 23, a health information screen 27 illustrated in FIG. 8 (S16: health-information displaying process). Herein, the health information screen 27 includes message images 230 to 232 and a cartoon character image 240 of a physician. The message image 230 indicates “advice for A”; who is the user. The message image 231 (first message) indicates the user's health condition, indicating “Hormone imbalance may have caused a bad condition”. The message image 232 (second message) is information for improving the user's health condition, indicating “Try to have deficient nutrients every day: Soy isoflavones, Vitamin E, and Folic acid”. Thus, by referring to the health information screen 27, the user is able to know their health condition. Furthermore, the user is able to know the nutrients to have by referring to the health information screen 27, thereby managing their health.

Note that the messages illustrated in FIG. 8 are examples, and the messages are not limited to these. For example, if menorrhagia is determined from the “menstruation information” and the napkin image, the message image 231 indicating the health condition may be “You have menorrhagia and may be anemic”. In such a case, the message image 232 may be “Supplement with iron”.

In addition, for example, the message image 231 may be a message about stress, indicating “You may be highly stressed”. In such a case, the message image 232 may be advice for relaxing the user and relieving the stress, such as “Take a slow deep breath”, “Hot yoga is recommended”, or “Listen to your favorite music”. In this manner, the message image 231 includes the user's health condition (e.g., stress, hormone, or condition), and the message image 232 includes advice for improving the user's health condition (e.g., meal, condition management, exercise, or method for relieving stress).

Although embodiments of the present disclosure have been described hereinabove, the above embodiments of the present disclosure are simply to facilitate understanding of the present disclosure and are not in any way to be construed as limiting the present disclosure. The present disclosure may variously be changed or altered without departing from its gist and encompass equivalents thereof. For example, modification which will be described below is possible.

In one or more embodiments, an image of a napkin to which menstrual blood is attached is captured, and the napkin image 210 is transmitted to the server 11, but without limitation to this. For example, it is acceptable that, while a user is not on a period, an image of a napkin to which “vaginal discharge” (menstrual blood, occult blood, etc. discharged from the vagina) of the user of the napkin is captured. In such a case, the image of the napkin to which vaginal discharge is attached is transmitted to the server 11, and thus, on the basis of information of vaginal discharge or the like, the server 11 can determine stress of the user or the like.

In addition, in step S15, the generating module 106 generates information indicating a message to the user on the basis of the determination data 90, but without limitation to this. For example, the server 11 (or a physician) may create the message for the user. In such a case, the determination data 90 includes the message images 231 and 232 for the user. Thus, on the basis of the determination data 90, the health-information displaying module 107 may display the health information screen 27 on the display 23.

In addition, on the input screen 26 displayed is the message image 224 that prompts the user to input whether there is any clotted matter in the vaginal discharge, but without limitation to this. For example, on the basis of the napkin image 210, the server 11 may determine whether there is any clotted matter in the vaginal discharge, by using image processing instructions.

In addition, on the input screen 26 displayed is the message image 227 for designating the napkin size and the percentage of the menstrual blood area, but without limitation to this. For example, on the basis of the napkin image 210, the server 11 may determine the percentage of the menstrual blood (area relative to the whole area), by using image processing instructions.

In addition, as in step S12, the message images are displayed sequentially on the input screen 26, but without limitation to this. For example, all of the message images 220 to 227 may be displayed on the display 23 at the same time.

In addition, information about the number of napkins used can be selected by using choices on the input screen 26, but without limitation to this. For example, the number of napkins used may be directly input on the display 23.

In addition, although the image capturing target in one or more embodiments is the “napkin” used by the user, other absorbent articles, such as a “liner” or “diaper” may also be used, for example.

The terminal 10 transmits three data items, which are the medical-interview data 40, the menses data 50, and the image data item 60, to the server 11, but without limitation to this. For example, at least the image data item 60 may be transmitted. In such a case, the server 11 determines the user's health condition on the basis of the image data item 60.

Furthermore, the program 30 is stored in the storage unit 20 of the terminal 10. Alternatively, for example, the program 30 may be stored on a medium (DVD: digital versatile disc) that stores data or the like and may be executed by a processor (not illustrated) of the terminal 10.

In addition, instructions may be supplied to a computer by using a non-transitory computer readable medium with an executable set of instructions thereon. Note that examples of the non-transitory computer readable medium include a magnetic recording medium (e.g., flexible disk, magnetic tape, or hard disk drive), a CD-ROM (read only memory), and the like.

Although the disclosure has been described with respect to only a limited number of embodiments, those skilled in the art, having benefit of this disclosure, will appreciate that various other embodiments may be devised without departing from the scope of the present invention.

Accordingly, the scope of the invention should be limited only by the attached claims.

### REFERENCE SIGNS LIST

10 terminal

11 server

20 storage unit

21 control unit

22 input unit

23 display

24 camera

26 input screen

27 health information screen

30 program

40 medical-interview data

50 menses data

60, 70 to 76 image data item

90 determination data

100 instruction displaying module

101 field-instruction displaying module

102 input-screen displaying module

103 menses-information receiving module

104 transmission module

105 reception module

106 generating module

107 health-information displaying module

200, 220, 222, 224 to 227, 230 to 232 message image

221, 223 image

201 field instruction image

210 napkin image

240 cartoon character image

What is claimed is:

1. A non-transitory computer readable medium storing executable instructions that cause a computer to execute:
transmitting, to a server, an image of an absorbent article containing vaginal discharge of a user;
receiving, from the server, health information that is obtained from the image and indicates a health condition of the user; and
displaying, on a display, a health information screen based on the health information, wherein
the health information screen displays a first message that indicates the health condition and a second message for improving the health condition.

2. The non-transitory computer readable medium according to claim 1, wherein
the vaginal discharge includes menstrual blood, and
the instructions cause the computer to further execute:
displaying, on the display, a third message that prompts the user to capture the image of the absorbent article when a predetermined number of days have passed since start of a menstruation of the user.

3. The non-transitory computer readable medium according to claim 2, wherein the third message prompts the user to capture the image of the absorbent article on a morning of a fourth day since the start of the menstruation, the absorbent article having been used overnight on a third day since the start of the menstruation.

4. The non-transitory computer readable medium according to claim 1, further comprising:
transmitting, to the server, user information of the user, wherein
the health information is obtained based on the image and the user information.

5. The non-transitory computer readable medium according to claim 1, wherein
the vaginal discharge includes menstrual blood,
the instructions cause the computer to further execute:
displaying, on the display, an input screen that receives menstruation information about menstruation at the time of capturing the image of the absorbent article;
receiving the menstruation information input via the display; and
transmitting, to the server, the menstruation information, and the health information is obtained based on the image and the menstruation information.

6. The non-transitory computer readable medium according to claim 1, wherein
the vaginal discharge includes menstrual blood,
the instructions cause the computer to further execute:
displaying, on the display, an input screen that receives menstruation information about menstruation at the time of capturing the image of the absorbent article;
receiving the menstruation information input via the display; and
transmitting, to the server, user information of the user and the menstruation information, and
the health information is obtained based on the image, the user information, and the menstruation information.

7. The non-transitory computer readable medium according to claim 5, wherein
the menstruation information includes information about a number of absorbent articles that have been used by the user before capturing the image of the absorbent article, and
the input screen displays a fourth message that prompts the user to input the number of absorbent articles.

8. The non-transitory computer readable medium according to claim 5, wherein
the menstruation information includes information about a color of the menstrual blood contained in the absorbent article, and
the input screen displays a plurality of colors and a fifth message that prompts the user to select, from among the colors, a darkest color of the menstrual blood contained in the absorbent article.

9. The non-transitory computer readable medium according to claim 5, wherein
the menstruation information about whether the vaginal discharge includes a clotted matter, and
the input screen displays a sixth message that prompts the user to input whether the vaginal discharge includes the clotted matter.

10. The non-transitory computer readable medium according to claim 1, wherein
the instructions cause the computer to further execute:
displaying, on the display, a field instruction image that prompts the user to capture an image of a to-be-imaged absorbent article that includes a portion containing no menstrual blood.

11. The non-transitory computer readable medium according to claim 1, wherein
the first message indicates stress or hormone balance.

12. The non-transitory computer readable medium according to claim 1, wherein
the second message indicates a nutrient that the user should take.

13. The non-transitory computer readable medium according to claim 1, wherein
the health information indicates stress or hormone balance,
the instructions cause the computer to further execute:
generating information about the first message and the second message based on the health information.

14. A method for health management support, comprising:
transmitting, by a computer to a server, an image of an absorbent article containing vaginal discharge of a user;
receiving, by the computer from the server, health information that is obtained based on information transmitted from the computer and indicates a health condition of the user; and
displaying, by the computer on a display, a health information screen based on the health information, wherein
the health information screen displays a first message that indicates the health condition and a second message for improving the health condition.

15. A computer system comprising:
a controller that:
transmits, to a server, an image of an absorbent article containing vaginal discharge of a user;
receives, from the server, health information that is obtained based on information transmitted from the controller and indicates a health condition of the user; and
displays, on a display, a health information screen based on the health information, wherein
the health information screen displays a first message that indicates the health condition and a second message for improving the health condition.
